(19) United States
(12) Patent Application Publication
(10) Pub. No.: US 2026/0018267 A1
(43) Pub. Date: Jan. 15, 2026

(54) AUTOMATED PATIENT CHARTING

(71) Applicant: Welch Allyn, Inc., Skaneateles Falls, NY (US)

(72) Inventors: WonKyung McSweeney, Manlius, NY (US); Allison J. DelCostello, Marietta, NY (US); Danielle R. Endres, Syracuse, NY (US); Corinn C. Fahrenkrug, Chittenango, NY (US); John A. Lane, Venice, FL (US); David E. Quinn, Moravia, NY (US); Gene J. Wolfe, Pittsford, NY (US)

(21) Appl. No.: 19/252,205

(22) Filed: Jun. 27, 2025

Related U.S. Application Data
(60) Provisional application No. 63/669,279, filed on Jul. 10, 2024.

(51) Int. Cl. G16H 10/60; G10L 15/26; G16H 50/30; G16H 70/20

(52) U.S. Cl. CPC G16H 10/60; G10L 15/26; G16H 50/30; G16H 70/20

(57) ABSTRACT

A system for capturing patient data. The system captures context data of the patient. The context data includes at least one of visual data captured by a camera and audio data captured by a microphone. The system generates a context based on the context data, retrieves one or more guidelines based on the context, sends the one or more guidelines to a machine learning model, and receives instructions from the machine learning model. The system captures the patient data using at least one of the camera and the microphone based on the instructions from the machine learning model. The system stores the patient data in an electronic medical record of the patient.

### CROSS-REFERENCE TO RELATED APPLICATION(S)

This application claims the benefit of and priority to U.S. Provisional Patent Application No. 63/669,279, filed Jul. 10, 2024, the entire disclosure of which is incorporated by reference herein in its entirety.

### BACKGROUND

A head-to-toe assessment is crucial in a medical-surgical setting for several key reasons such as performing a comprehensive evaluation to provide a complete overview of a patient's condition allowing for early detection of potential complications; establishing baseline information to enable healthcare providers to monitor progress or deterioration over time; contributing to improved patient safety by identifying risks such as pressure ulcers; informing decision-making to ensure that care plans are tailored to individual patient needs; and providing effective communication between healthcare team members promoting continuity of care.

However, documenting head-to-toe assessments presents several challenges. Nurses face time constraints with multiple patients and limited time for thorough documentation. Attention to detail is crucial as assessments involve specifics, making it easy to overlook important information. Additionally, patients' conditions can change rapidly, necessitating the need to keep records current. Electronic Health Records (EHR) systems can be complex and challenging to navigate, slowing down the documentation process. Maintaining privacy and accuracy is paramount to avoid potential issues caused by mistakes. The physical strain of constant movement within a healthcare setting such as a floor of a hospital can cause fatigue among nurses, which can impact their focus on proper documentation. Effective communication is essential to prevent errors in records. Less experienced nurses may struggle with efficient documentation, highlighting the importance of training and experience. Consistency in documentation methods across different settings can be confusing, while language barriers between staff and patients can complicate assessments and documentation.

Further complicating the matter, nursing shortages increase workloads and stress on presently employed nurses. The root causes of nursing shortages range from an aging nursing workforce, high attrition rates due to burnout, and a growing demand for healthcare services especially in light of an aging population in the developed world. As a result, nursing shortages present further obstacles to documenting head-to-toe assessments in clinical settings.

### SUMMARY

In general terms, the present disclosure relates to automated patient charting. In one possible configuration, outputs from a machine learning model are enhanced for capturing patient data used in generating the patient charting. Various aspects are described in this disclosure, which include, but are not limited to, the following aspects.

One aspect relates to a system for capturing patient data, the system comprising: a processing device; and a computer readable data storage device storing software instructions that, when executed by the processing device, cause the system to: capture context data of the patient, the context data including at least one of visual data captured by a camera and audio data captured by a microphone; generate a context based on the context data; retrieve one or more guidelines based on the context; send the one or more guidelines to a machine learning model; receive instructions from the machine learning model; capture the patient data using at least one of the camera and the microphone based on the instructions from the machine learning model; and store the patient data in an electronic medical record of the patient.

Another aspect relates to a method for capturing patient data, the method comprising: capturing context data of the patient, the context data including at least one of visual data captured by a camera and audio data captured by a microphone; generating a context based on the context data; retrieving one or more guidelines based on the context; sending the one or more guidelines to a machine learning model; receiving instructions from the machine learning model; capturing the patient data using at least one of the camera and the microphone based on the instructions from the machine learning model; and storing the patient data in an electronic medical record of the patient.

A variety of additional aspects will be set forth in the description that follows. The aspects can relate to individual features and to combination of features. It is to be understood that both the foregoing general description and the following detailed description are exemplary and explanatory only and are not restrictive of the broad inventive concepts upon which the embodiments disclosed herein are based.

### DESCRIPTION OF THE FIGURES

The following drawing figures, which form a part of this application, are illustrative of the described technology and are not meant to limit the scope of the disclosure in any manner.

FIG. 1 illustrates an example of a system that can perform various assessments of a patient with and/or without a nurse present.

FIG. 2 schematically illustrates an example of a method of performing an assessment of the patient using the system of FIG. 1.

FIG. 3 schematically illustrates an example of a method of updating a vector database to improve chartable data generated in accordance with the method of FIG. 2.

FIG. 4 schematically illustrates an example of a method of performing a head-to-toe assessment of the patient that can be performed by the system of FIG. 1.

FIG. 5 schematically illustrates an example of a method of processing visual and audio data captured during performance of the method of FIG. 4.

FIG. 6 schematically illustrates an example of a computing device that can be used to implement aspects of the system of FIG. 1.

### DETAILED DESCRIPTION

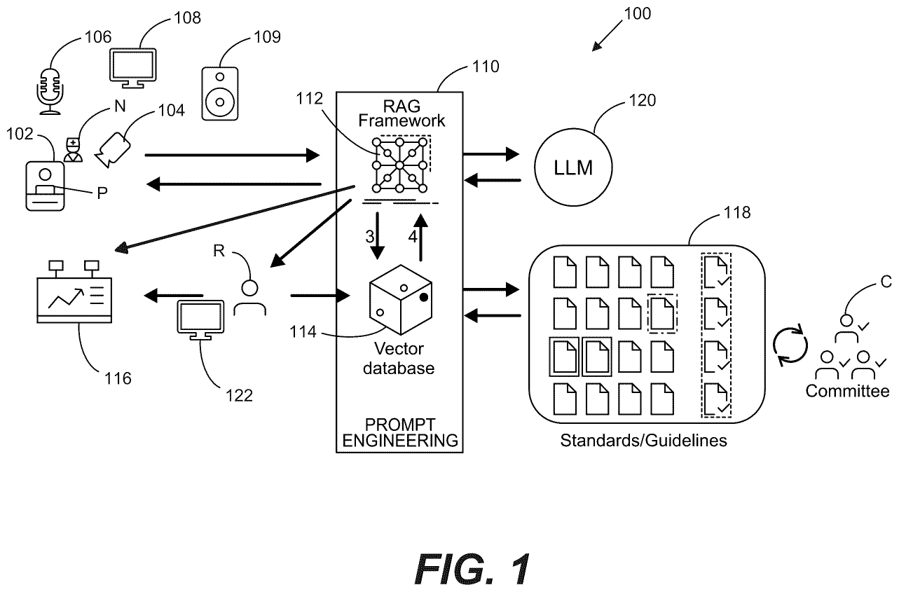

FIG. 1 illustrates an example of a system 100 that can perform various assessments of a patient P with and/or without a nurse N present. The assessments performed by the system 100 can be worked into existing clinical workflows to save time that would otherwise be spent by the nurse N on documentation and charting. Further, the assessments performed by the system 100 can provide objective observations of the patient P to reduce guess work for types of physical assessments that are typically performed by the nurse N throughout the day such as during rounding. In some instances, the system 100 can assist the nurse N throughout a head-to-toe assessment of the patient P such as in a medical-surgical setting. In further examples, the system 100 performs pain assessments, neurological assessments including stroke assessments, and other types of assessments when the nurse N is not physically present next to the patient P.

In FIG. 1, a patient P is shown resting on a patient support apparatus 102. Illustrative examples of the patient support apparatus 102 include a hospital bed, a stretcher, a surgical table, or any other apparatus on which a patient can rest while being physically assessed.

The system 100 further includes a camera 104 that captures a video stream of the patient P and their surroundings which may include the nurse N such as when the nurse N is physically present next to the patient P such as when the nurse N is interacting with the patient P. In some examples, the camera 104 can be worn by the nurse N. In some instances, the system 100 includes more than the one camera shown in FIG. 1 such that the system 100 may include multiple cameras for capturing multiple video streams such as from different perspectives or imaging modalities. For example, the multiple cameras can each have a different imaging modality including, without limitation, a camera for capturing light in red, green, and blue (RGB) wavelengths, a camera that provides both depth (D) and color (RGB) data as an output in real-time (RGB-D), a camera that captures infrared (IR) images, a pan-tilt-zoom (PTZ) camera, and other types of modalities for capturing relevant medical data.

The system 100 further includes a microphone 106 that captures audio of the patient P and their surroundings. For example, the microphone can capture a conversation between the patient P and the nurse N such as when the nurse N asks the patient questions as part of a physical assessment of the patient or that are otherwise medically relevant. The system 100 can further include a speaker 109 that can emit audio prompts, as described further below.

In further examples, the system 100 can include additional sensors to measure parameters such as grip strength. Further, the system 100 can include one or more probes that can be used to provide stimuli such as percussion of the patient P's body parts for assessment of pain. Such sensors can be built into the patient support apparatus 102. For example, pressure sensors that measure the grip strength and the one or more probes can be integrated into one or more rails of the patient support apparatus 102. The patient support apparatus 102 can share similar aspects with the patient support apparatus described in U.S. patent application Ser. No. 17/368,095, filed Jul. 6, 2021, the disclosure of which is herein incorporated by reference in its entirety. Alternatively, such sensors can be included on an autonomous robot.

The system 100 can further include a display monitor 108 that can be used during the physical assessment of the patient P. For example, the display monitor 108 can be used to display instructions for either the patient P or the nurse N to perform during the physical assessment. The display monitor 108 may also be used to display a checklist and/or guidelines for the nurse N to follow when performing the physical assessment of the patient P. In some instances, the display monitor 108 is a television (TV) that can be used to provide entertainment to the patient P when not being used during the physical assessment. The display monitor 108 can be mounted on a wall, on a mobile cart that can moved in proximity to the patient P, or can be attached to another device or piece of equipment such as the patient support apparatus 102. In further examples, the display monitor 108 is a portable computing device such as a tablet device or smartphone.

In some examples, the patient support apparatus 102, the camera 104, the microphone 106, and the display monitor 108 are located within a designated area of a healthcare facility such as a patient room of a hospital. In such examples, these devices can be used to monitor the patient P while the patient P is located and/or admitted to the patient room.

As further shown in FIG. 1, the system 100 includes a server 110 that includes a retrieval augmented generator (RAG) model 112 and a vector database 114 that stores vectors associated with a plurality of guidelines 118. The guidelines 118 can be created by a committee C of caregivers and/or administrators employed by the healthcare facility such as a hospital where the system 100 is operational. The RAG model 112 enhances the accuracy and reliability of generative artificial intelligence (AI) models including a large language model (LLM) 120 by retrieving data from external sources such as from the plurality of guidelines 118 and an electronic medical record (EMR) system 116 that includes an EMR of the patient P.

In the example shown in FIG. 1, the data retrieved by the RAG model 112 enhances the LLM 120 used by the system 100 to facilitate assessments of the patient P. The LLM 120 is an example of a neural network that includes parameters that represent the general patterns of how humans use words to form sentences. The RAG model 112 fills gaps within the LLM 120 by giving sources that the LLM 120 can cite so that the nurse N can verify any claims made by the LLM 120 to build trust in the outputs that are generated by the LLM 120. Further, the RAG model 112 helps the LLM 120 to clear up ambiguities in queries received from the nurse N and/or the patient P, and can also reduce the possibility that the LLM 120 will make a wrong guess, a phenomenon sometimes called hallucination.

As an illustrative example, the RAG model 112 can connect the LLM 120 to the plurality of guidelines 118 that are established by the committee C of the healthcare facility where the system 100 is operational. In such examples, when the nurse N asks the LLM 120 a question, the LLM 120 sends the query to an RAG model 112 that converts the question into a numeric format allowing machines to read it. The numeric version of the query is called a vector. The RAG model 112 compares the numeric values to vectors in a vector database 114, which is a machine-readable index of an available knowledge base. When the RAG model 112 finds a match or multiple matches, it retrieves the related data, converts it to human-readable words and passes it back to the LLM 120. Then, the LLM 120 combines the retrieved words and its own response to the query into a final answer it presents to the user, potentially citing the guidelines 118 found by the RAG model 112. In the background, the RAG model 112 continuously updates the vector database 114, for new and updated knowledge bases as they become available.

In alternative examples, instead of utilizing the vector retrieval performed by the RAG model 112, the system 100 can utilize additional types of data structures such as knowledge graph-based retrieval to supply the LLM 120 with relevant information for enhancing the outputs of the LLM 120.

As further shown in FIG. 1, the system 100 includes a display monitor 122 that displays chartable data generated by the server 110. The chartable data is displayed on the display monitor 122 for review by a reviewer R such as a remote caregiver or technician who reviews the correctness of the chartable data. In alternative examples, the reviewer R can include the nurse N such as after the nurse finishes visiting the patient P in the designated area of the healthcare facility (e.g., patient room in hospital), or when the nurse N completes rounding for multiple patients. When the server 110 receives confirmation from the reviewer R, the server 110 charts the chartable data such as by uploading the chartable data to the EMR of the patient P.

The system 100 helps to address limitations resulting from nursing shortages by improving nursing efficiencies. The system 100 improves nursing efficiencies by providing the nurse N with advanced tools for automated execution of nursing tasks such as patient charting and documentation more efficiently. This can mitigate nurse burnout by reducing task burdens on the nurse N. Further, the advanced tools provided by the system 100 eliminate time that would otherwise be spent by the nurse N away from the patient P, and thereby increases opportunities for the nurse N to provide hands-on care to improve patient outcomes.

The system 100, by using the RAG model 112 in combination with the LLM 120, provides computer vision as part of a charting information source, which opens up the scope of the automated charting to more care environments and more events that can be automated. Further the system 100 includes prompt engineering that allows different workflows and charting templates for different healthcare contexts without having to fine tune or re-train the LLM 120.

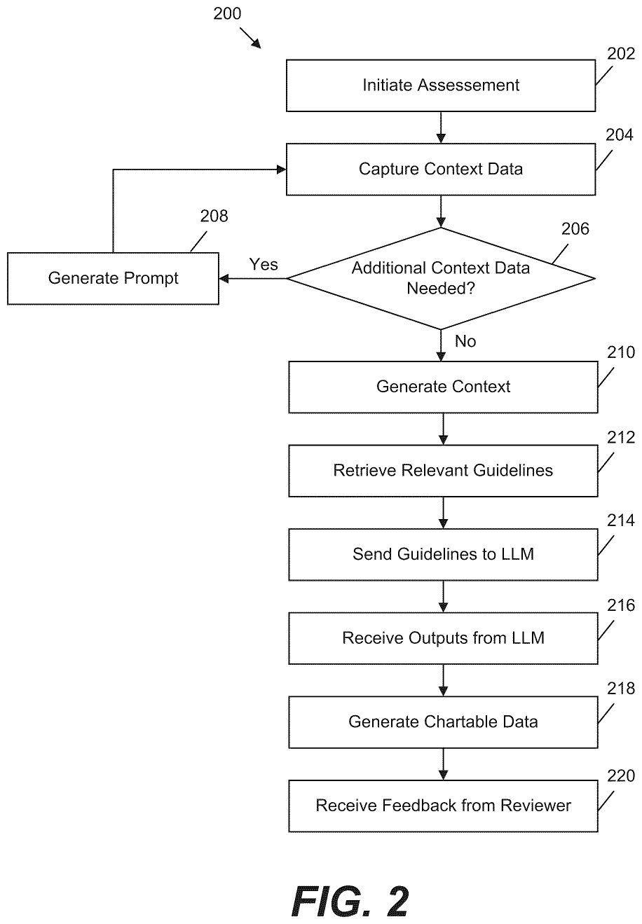

FIG. 2 schematically illustrates an example of a method 200 of performing an assessment of the patient P using the system 100. The method 200 includes an operation 202 of initiating the assessment of the patient P. Operation 202 can include initiating the assessment based on a schedule set in advance for the patient P such as upon admission to the healthcare facility. In some examples, the assessment is initiated when the nurse N is not present.

Alternatively, operation 202 can include initiating the assessment when either the camera 104 or the microphone detect the presence of the nurse N in proximity to the patient P, or when the nurse Nutters a verbal cue (e.g., “start assessment”) or physically selects an option to initiate the assessment such as by selecting an icon displayed on the display monitor 108.

The method 200 includes an operation 204 of capturing context data of the patient P. The context data can include visual data captured by the camera 104 (e.g., a grimace on the patient P's face in response to stimuli) and audio data captured by the microphone 106 (e.g., groans from the patient P or a conversation between the nurse N and the patient P).

The method 200 includes an operation 206 of determining whether additional context data is needed by the system 100 to generate a context for the assessment of the patient P. When additional context data is needed (i.e., “Yes” in operation 206), the method 200 proceeds to an operation 208 of generating a prompt for capturing additional context data. The prompt can include an instruction for the patient P or the nurse N to perform. As an illustrative example, the prompt can request the nurse N to reposition themself or the patient P, or to reposition an object for the camera 104 to capture an unobstructed video stream of the patient P. The prompt can be visually displayed on the display monitor 108 and/or can include an audio instruction emitted by the speaker 109 positioned in proximity to the patient P and the nurse N.

When additional context data is not needed (i.e., “No” in operation 206), the method 200 proceeds to an operation 210 of generating a context for the assessment of the patient P. As an illustrative example, the context can include a status of the patient P such as whether the patient P is experiencing pain, has finished eating a meal, or has returned from the bathroom.

In operation 210, the context is generated in a text format. For example, the LLM 120 can convert the visual data captured by the camera 104 and the audio data captured by the microphone 106 into a text format that can be used by the RAG model 112.

The method 200 includes an operation 212 of retrieving one or more guidelines 118 for the assessment of the patient P based on the context generated in operation 210. In some examples, in operation 212, the RAG model 112 uses the context generated in text format to generate a vector. The RAG model 112 then matches the vector based on the context with one or more vectors stored in the vector database 114 for selection of the one or more guidelines 118. In alternative examples, operation 212 can include using additional types of data structures such as knowledge graph-based retrieval to retrieve the one or more guidelines 118 for the assessment of the patient P based on the context generated in operation 210.

The method 200 includes an operation 214 of sending the one or more guidelines 118 together with the visual data captured by the camera 104 and the audio data captured by the microphone 106 to the LLM 120. In operation 214, the one or more guidelines 118, the visual data, and the audio data are sent in a predefined prompt format for use by the LLM 120.

The predefined prompt format can be configured by the healthcare facility per their own protocol. The predefined prompt format can include a basic prompt such as “As a registered nurse caring for a patient in XX condition, perform Y assessment,” or can include more context with some patient specific information such as “As a nurse caring for a patient, perform Y test for someone who has just gone through a knee surgery X hours ago.”

The method 200 includes an operation 216 of receiving outputs from the LLM 120. The outputs from the LLM 120 include strings of words that form one or more sentences such as instructions for the nurse N or the patient P to follow to capture chartable data for completion of the assessment of the patient P. As described above, the RAG model 112 fills gaps and/or clears up ambiguities in the outputs from the LLM 120 by giving sources such as the EMR 116 and the guidelines 118 that the LLM 120 can cite allowing the nurse N to verify the outputs made by the LLM 120. As an illustrative example, the outputs from the LLM 120 can include an instruction for the patient P to remain still while a physiological parameter (e.g., blood pressure) is being measured. As another example, the outputs from the LLM 120 can include an instruction for the patient P to show a body part (e.g., a limb) that needs to be examined as part of the assessment.

The outputs from the LLM 120 can be displayed on the display monitor 108 and/or can be emitted as audio from the speaker 109. The outputs from the LLM 120 are received while the assessment of the patient P is being performed to guide the assessment such that the assessment follows one or more guidelines 118 that are appropriate for the patient P's context.

The method 200 includes an operation 218 of generating chartable data based on the visual data captured by the camera 104 and the audio data captured by the microphone 106. The chartable data is generated while the outputs from the LLM 120 are being provided to guide the assessment of the patient P to follow the one or more guidelines 118 appropriate for the patient P's context. In operation 218, the chartable data can be displayed on the display monitor 122 for review by the reviewer R prior to being uploaded to the EMR of the patient P.

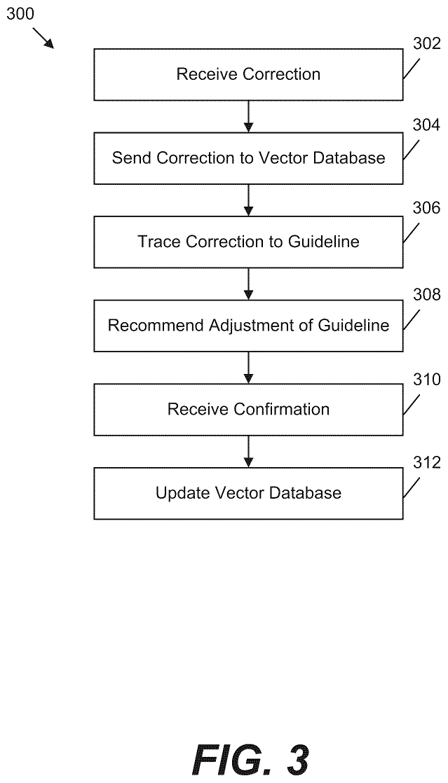

The method 200 includes an operation 220 of receiving feedback from the reviewer R. For example, when the reviewer R confirms or approves the chartable data generated in operation 218, the chartable data can be uploaded and/or stored in the EMR of the patient P. Alternatively, when the chartable data is rejected or modified by the reviewer R, the system 100 can update the vector database 114, as will not be described with reference to FIG. 3.

FIG. 3 schematically illustrates an example of a method 300 of updating the vector database 114 to improve the chartable data generated in accordance with the method 200 of FIG. 2. The method 300 can be performed by the system 100 when the feedback received in operation 220 of the method 200 identifies a correction to be made to the chartable data.

As shown in FIG. 3, the method 300 includes an operation 302 of receiving a correction of the chartable data generated by the system 100. As an illustrative example, the correction can identify a physiological parameter relevant for assessing progress or deterioration of a disease state (e.g., sepsis) that is missing from the chartable data.

The method 300 includes an operation 304 of sending the correction received in operation 302 to the vector database 114. The method 300 can include an operation 306 of tracing the correction to a portion of the one or more guidelines 118 retrieved by the RAG model 112 in operation 212 of the method 200. Thereafter, the method 300 includes an operation 308 of recommending an adjustment to the one or more guidelines 118 such as to prevent or mitigate future occurrences of the correction received from the reviewer in operation 302.

The method 300 can then include an operation 310 of receiving a confirmation or approval of the recommendation by the committee C of caregivers and/or administrators employed by the healthcare facility where the system 100 is operational. Thereafter, the method 300 can include an operation 312 of updating the vector database 114 based on the recommendation confirmed or approved by the committee C. In this manner, the method 300 can continuously improve the vector database 114 to ensure that the guidelines 118 are up-to-date and facilitate capture of accurate and reliable chartable data for assessment of the patient P.

In instances where the one or more guidelines 118 are accurate and sufficient, but the retrieval match by the RAG model 112 in the vector database 114 is the source of the error for the correction, operation 312 can include updating a prompt template to improve the retrieval of the one or more guidelines from the vector database by the RAG model 112. For example, when the prompt template does not match well with a guideline, the terms and expression of the prompt template can be changed to improve the vector similarity score where the right part of the guideline is retrieved and used for the assessment. As an illustrative example, when an initial prompt template is defined as “assess patient who had a surgery 24 hours ago”, and when the guidelines for different types of surgery vary widely, the retrieval of a guideline by the RAG model 112 will not be optimal. In such example, the prompt template can be updated to include more context or specificity such as “patient who had a knee surgery within 24 hours”.

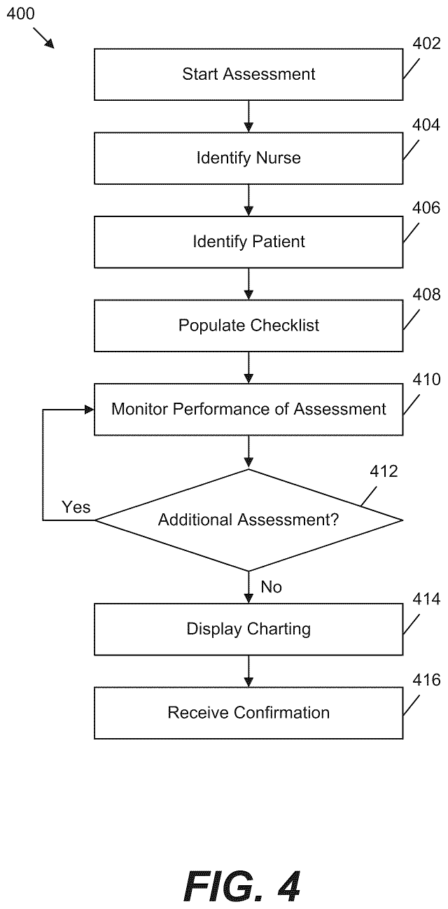

FIG. 4 schematically illustrates an example of a method 400 of performing a head-to-toe assessment of the patient P that can be performed by the system 100. In some examples, the method 400 is performed at admission to the healthcare facility to generate a baseline for the patient P against which patient P's healing progression or deterioration can be measured. As shown in FIG. 4, the method 400 includes an operation 402 of starting the head-to-toc assessment. In some examples, operation 402 is triggered by the nurse N uttering a wake word (e.g., “start assessment”) or by making a gesture with a body part (e.g., waving their hand).

The method 400 includes an operation 404 of identifying the nurse and an operation 406 of identifying the patient P. Operations 404, 406 are performed simultaneously, or substantially at the same time. Operations 404, 406 can include using the visual data captured by the camera 104 to detect ID badges worn by the nurse N and/or patient P to identify the nurse N and the patient P, respectively. Alternatively, facial recognition analysis can be performed on the visual data to identify the nurse N and/or the patient P. In yet further examples, the audio data captured by the microphone 106 can be analyzed to identify the nurse N and/or the patient P. Additional examples for identifying the nurse N and patient P based on the visual data and audio data captured by the camera 104 and the microphone 106, respectively, are possible.

The method 400 includes an operation 408 of populating a checklist that includes tasks for performing the head-to-assessment of the patient P. The checklist can be populated based on a condition or diagnosis of the patient P which can be determined from the patient P's EMR. For example, the checklist is modified to include tasks that are specific for identifying or evaluating sepsis when the patient P is identified in the EMR as being at risk for sepsis. The checklist including the tasks can be displayed on the display monitor 108.

The method 400 includes an operation 410 of monitoring performance of the assessment using the visual data captured by the camera 104 and the audio data captured by the microphone 106. Operation 410 can include tracking movements of both the patient P and the nurse N with a pre-configured head to assessment protocol. As the nurse N performs each task in the checklist populated in operation 408, the system 100 creates structured data to be charted for the head-to-toc assessment of the patient P. The inputs for the structured data can include the voice of the nurse N captured by the microphone 106 and the video data captured by the camera 104. input. As an illustrative example, when the nurse N touches the patient P's abdomen and the patient P expresses pain, the system 100 automatically suggests charting the information, or the nurse N can say out loud “pain in abdominal area” which is recorded by microphone 106.

As the steps of the head-to-toe assessment are performed in operation 410, the system 100 can control the camera 104 to take visual snapshots or video clippings of medically relevant regions of interest. As an illustrative example, when the nurse N finds an existing ulcer on patient P's back, the nurse N can turn the patient P to have the ulcer visible for the camera 104, and the camera 104 can identify that there is a medically relevant region of interest such that the camera 104 zooms into the region of interest to capture an image of the ulcer with other relevant data such as a verbal description of the ulcer uttered by the nurse N.

In this manner, when the head-to-assessment is repeated, the images of the region of interest and other relevant data can be compared over time to indicate a trend such as whether a condition in the region of interest (e.g., ulcer) is improving or getting worse. During admission to the healthcare facility, this information can be used by the system 100 to prevent worsening. For example, when the region of interest (e.g., ulcer site) is constantly touching the patient support apparatus 102 (e.g., hospital bed), the system 100 can recommend turning the patient P.

The method 400 includes an operation 412 of determining whether additional tasks remain to be performed for completion of the head-to-toe assessment of the patient P. As the nurse N goes through the tasks listed in the checklist populated in operation 408, the system 100 keeps track of what tasks have been completed and what tasks have not been completed. The nurse N can indicate completion of the tasks via voice (e.g., “XXX normal”, “YYY normal”), or the nurse N can perform the tasks and the system 100 can recognize the tasks from analysis of the visual and/or audio data captured by the camera 104 and the microphone 106, respectively, and the system 100 can check off the recognized tasks when they are completed. In some instances, the system 100 can also provide audio feedback through the speaker 109 to indicate to the nurse N the relevant health data that is being charted or captured by the system 100.

When operation 412 determines that there are additional tasks that need to be performed for completion of the head-to-toe assessment of the patient P (i.e., “Yes” in operation 412), the method 400 can return to operation 410. The checklist can be updated to show the remaining tasks that need to be completed for the head-to-toe assessment.

When operation 412 determines that there are no additional tasks that need to be performed for completion of the head-to-toe assessment of the patient P (i.e., “No” in operation 412), the method 400 proceeds to an operation 414 of displaying charted data that is based on the monitoring of the tasks performed in operation 410. Operation 414 can include displaying the chartered data in a user interface displayed proximate the patient P such as bedside. For example, operation 414 can include displaying the charted data on the display monitor 108. Operation 414 can include displaying a review screen that shows evidence of the charted data (e.g., an image of an ulcer captured by the camera 104). Further, when the head-to-toe assessment is repeated, operation 414 can include displaying a progression of the charted data over time such as whether new health problems are identified, or previously identified health problems are worsening.

The method 400 includes an operation 416 of receiving a confirmation of the charted data by the nurse N. For example, the nurse N can review the charted data displayed in operation 414, and can submit a confirmation or approval of the charted data. In instances when the nurse N disagrees with an evaluation or assessment of a task in the charted data, the nurse N can override and/or modify the charted data. When confirmation or approval is received in operation 416, the system 100 can upload or store the charted data in the EMR of the patient P.

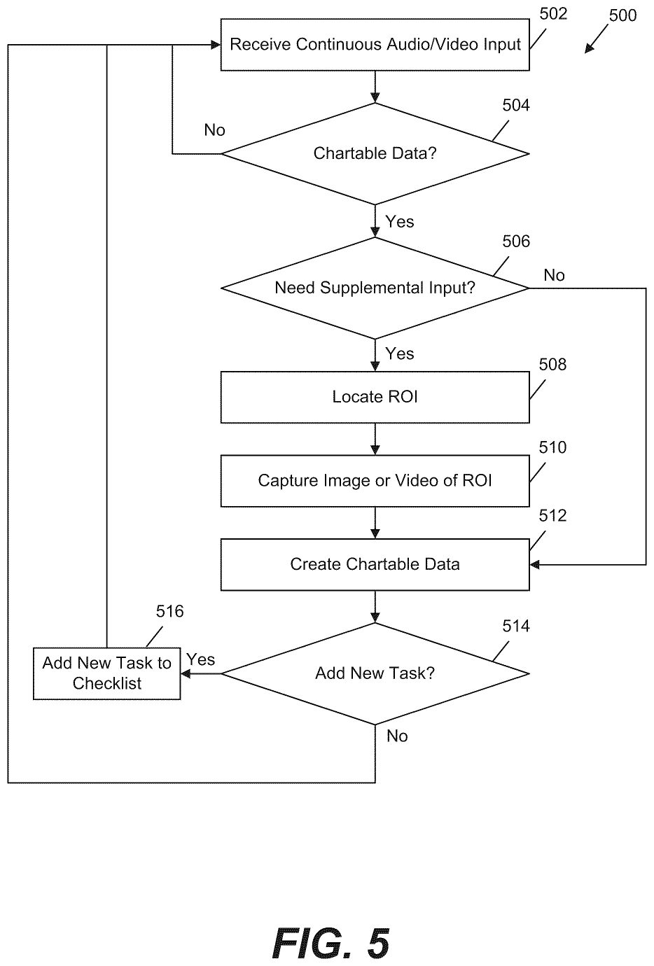

FIG. 5 schematically illustrates an example of a method 500 of processing the visual and audio data captured during monitoring of the tasks performed in operation 410 of the method 400. The method 500 includes an operation 502 of receiving a continuous stream of the visual data and the audio data captured by the camera 104 and the microphone 106, respectively.

The method 500 includes an operation 504 of determining whether the visual data and the audio data include chartable data. Operation 504 can include analyzing the voice of the nurse N using the LLM 120 to determine whether the stream of the visual data and the audio data includes chartable data. As an illustrative example, when the nurse N utters “pressure ulcer”, the LLM 120 analyzes the voice of the nurse N and determines that the nurse N is assessing a pressure ulcer of the patient P. Alternatively, or additionally, the system 100 can analyze the visual data captured by the camera 104 to recognize that medically relevant condition exists such as a pressure ulcer exposed on the patient P's body. When the visual data and the audio data does not include chartable data (i.e., “No” in operation 504), the method 500 returns to operation 502 and continues receiving the continuous stream of the visual data and the audio data.

When the visual data and the audio data does include chartable data (i.e., “Yes” in operation 504), the method 500 proceeds to an operation 506 of determining whether supplemental input is needed. When supplemental input is not needed (i.e., “No” in operation 506), the method 500 proceeds to an operation 512, which is described further below.

When supplemental input is needed (i.e., “Yes” in operation 506), the method 500 proceeds to an operation 508 of locating a region of interest. For example, the region of interest can include a medically relevant condition such as an injury such as a pressure ulcer. Operation 508 can include using computer vision techniques that can include machine learning and convolutional neural network (CNN) algorithms for identifying and locating the region of interest. In examples where the camera 104 is a pan-tilt-zoom (PTZ) camera, operation 508 can include panning, tilting, and zooming the camera 104 to focus on the region of interest.

The method 500 includes an operation 510 of capturing an image or a video of the region of interest using the camera 104. For example, operation 510 can include capturing an image or a video of the pressure ulcer when exposed on the patient P's body.

Next, the method 500 includes the operation 512 of creating chartable data that includes data extracted from the continuous stream of the visual data and the audio data as well as the image or video captured in operation 510. For example, operation 512 can include adding commentary from the nurse N to annotate the image or video captured in operation 510.

In some examples, the method 500 can include an operation 514 of determining whether a new task should be added to the checklist for performing the head-to-assessment of the patient P. The determination in operation 514 can be based on the chartable data created in operation 512. For example, when the chartable data identifies a new health condition that is not previously identified in the EMR of the patient P (i.e., “Yes” in operation 514), the method 500 proceeds to an operation 516 of adding a new task to the checklist to further investigate or assess the new health condition. Otherwise, when the chartable data does not identify a new health condition (i.e., “No” in operation 514), the method 500 returns to operation 502.

In some examples, the patient context can be determined based on the EMR of the patient. For example, a time-based retrieval from the EMR of the patient can be performed. As an illustrative example, a guideline can include “when the patient's blood pressure goes up by more than Y after medication X was given, do Z” such that the system 100 is triggered to do a time-based retrieval from the EMR of the patient. The system 100 can use meta-data within the EMR of the patient to perform auto-retrieval and time-based retrieval from the EMR of the patient, which are structured retrieval methods that can be performed by the system 100.

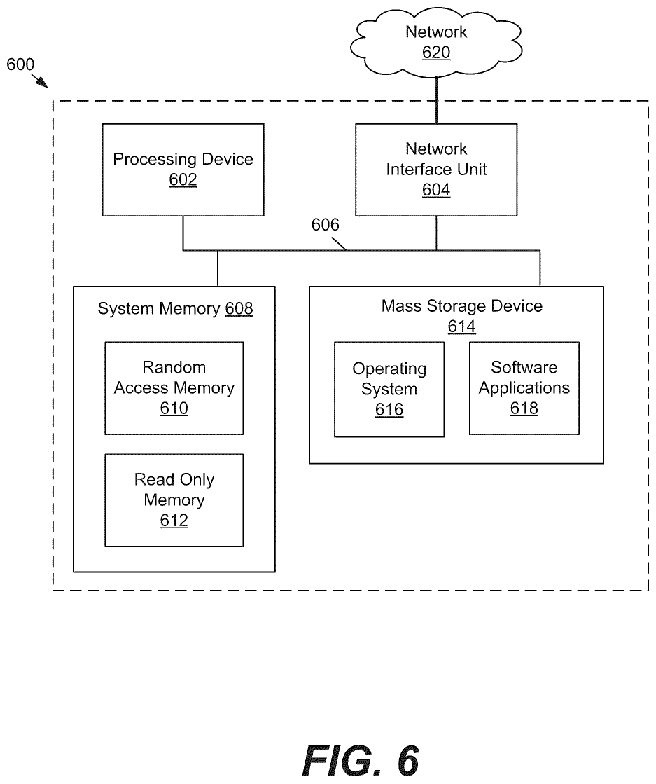

FIG. 6 schematically illustrates an example of a computing device 600 that can be used to implement aspects of the system 100. The computing device 600 includes at least one processing device 602, a system memory 608, and a system bus 606 that couples the system memory 608 to the at least one processing device 602. Further, the computing device 600 operates in a networked environment using logical connections to devices through the network 620. The computing device 600 connects to the network 620 through a network interface unit 604 connected to the system bus 606. The network interface unit 604 can also connect to other types of communications networks and devices, including through Bluetooth and Wi-Fi.

The at least one processing device 602 is an example of a processing unit such as a central processing unit (CPU). The at least one processing device 602 can include one or more CPUs. In some examples, the at least one processing device 602 includes one or more digital signal processors, field-programmable gate arrays, and/or other types of electronic circuits.

The system memory 608 includes a random-access memory (“RAM”) 610 and a read-only memory (“ROM”) 612. Basic input/output logic containing routines to transfer information between elements within the computing device 600 is stored in the ROM 612.

The computing device 600 can also include a mass storage device 614 that is able to store software instructions and data. The mass storage device 614 is connected to the at least one processing device 602 through a mass storage controller connected to the system bus 606. The mass storage device 614 and its associated computer-readable data storage media provide additional non-volatile, non-transitory storage for the computing device 600.

The mass storage device 614 and/or the system memory 608 can store software instructions and data. The software instructions can include an operating system 616 suitable for controlling the operation of the computing device 600. The mass storage device 614 and/or the system memory 608 also store software instructions 618, that when executed by the at least one processing device 602, cause the device to provide the functionality discussed herein.

Although the description of computer-readable data storage media contained herein refers to a mass storage device, it should be appreciated by those skilled in the art that computer-readable data storage media can be any available non-transitory, physical device or article of manufacture from which the device can read data and/or instructions. In certain embodiments, the computer-readable storage media comprises entirely non-transitory media. The mass storage device 614 is an example of a computer-readable storage device.

Computer-readable data storage media include volatile and non-volatile, removable and non-removable media implemented in any method or technology for storage of information such as computer-readable software instructions, data structures, program modules or other data. Example types of computer-readable data storage media include, but are not limited to, RAM, ROM, EPROM, EEPROM, flash memory or other solid-state memory technology, or any other medium which can be used to store information, and which can be accessed by the device.

The various embodiments described above are provided by way of illustration only and should not be construed to be limiting in any way. Various modifications can be made to the embodiments described above without departing from the true spirit and scope of the disclosure.

What is claimed is:

1. A system for capturing patient data, the system comprising:
a processing device; and
a computer readable data storage device storing software instructions that, when executed by the processing device, cause the system to:
capture context data of the patient, the context data including at least one of visual data captured by a camera and audio data captured by a microphone;
generate a context based on the context data;
retrieve one or more guidelines based on the context;
send the one or more guidelines to a machine learning model;
receive instructions from the machine learning model;
capture the patient data using at least one of the camera and the microphone based on the instructions from the machine learning model; and
store the patient data in an electronic medical record of the patient.

2. The system of claim 1, wherein retrieve the one or more guidelines based on the context includes:
converting the context into a text format;
generating a vector based on the text format; and
matching the vector with one or more vectors stored in a vector database.

3. The system of claim 2, wherein a retrieval augmented generator model retrieves the one or more guidelines from the vector database.

4. The system of claim 3, wherein the machine learning model is a large language model, and wherein the retrieval augmented generator model sends the one or more guidelines to the large language model in a predefined prompt format.

5. The system of claim 2, wherein the instructions, when executed by the at least one processing device, further cause the system to:
update a prompt template for retrieval of the one or more guidelines when the matching in the vector database is a source of error.

6. The system of claim 1, wherein the instructions, when executed by the at least one processing device, further cause the system to:
generate a prompt for capturing additional context data for generating the context.

7. The system of claim 1, wherein an assessment of the patient is automatically initiated based on a schedule set in advance for the patient, and wherein the assessment is guided by the one or more guidelines.

8. The system of claim 1, wherein an assessment of the patient is automatically initiated when the context data detects a presence of a nurse in proximity to the patient, and wherein the assessment is guided by the one or more guidelines.

9. The system of claim 1, wherein an assessment of the patient is initiated based on a verbal cue or a selection on a display monitor, and wherein the assessment is guided by the one or more guidelines.

10. The system of claim 1, wherein the instructions, when executed by the at least one processing device, further cause the system to:
receive a correction of the patient data;
trace the correction to a portion of the one or more guidelines; and
generate a recommendation for adjustment to the one or more guidelines to mitigate future occurrences of the correction of the patient data.

11. The system of claim 10, wherein the instructions, when executed by the at least one processing device, further cause the system to:
update a vector database based on the recommendation, the vector database being used for retrieving the one or more guidelines.

12. The system of claim 10, wherein the instructions, when executed by the at least one processing device, further cause the system to:
update a prompt template based on the recommendation to improve retrieval of the one or more guidelines from a vector database by a retrieval augmented generator model.

13. A method for capturing patient data, the method comprising:
capturing context data of the patient, the context data including at least one of visual data captured by a camera and audio data captured by a microphone;
generating a context based on the context data;
retrieving one or more guidelines based on the context;
sending the one or more guidelines to a machine learning model;
receiving instructions from the machine learning model;
capturing the patient data using at least one of the camera and the microphone based on the instructions from the machine learning model; and
storing the patient data in an electronic medical record of the patient.

14. The method of claim 13, wherein retrieving the one or more guidelines includes:
converting the context into a text format;
generating a vector based on the text format; and
matching the vector with one or more vectors stored in a vector database.

15. The method of claim 14, further comprising:
using a retrieval augmented generator model to retrieve the one or more guidelines from the vector database.

16. The method of claim 15, wherein the machine learning model is a large language model, and the method further comprising:
using the retrieval augmented generator model to send the one or more guidelines to the large language model in a predefined prompt format.

17. The method of claim 14, further comprising:
updating a prompt template for retrieval of the one or more guidelines when the matching in the vector database is a source of error.

18. The method of claim 13, further comprising:
receiving a correction of the patient data;
tracing the correction to a portion of the one or more guidelines; and
generating a recommendation for adjustment to the one or more guidelines to mitigate future occurrences of the correction of the patient data.

19. The method of claim 18, further comprising:
updating a vector database based on the recommendation, the vector database being used for retrieving the one or more guidelines.

20. The method of claim 18, further comprising:
updating a prompt template based on the recommendation to improve retrieval of the one or more guidelines from a vector database by a retrieval augmented generator model.
